(19) United States
(12) Patent Application Publication
(10) Pub. No.: US 2025/0000368 A1
(43) Pub. Date: Jan. 2, 2025

(54) POWER-CONSERVING SECURE SYSTEMS FOR VITAL SIGN MONITORING

(71) Applicant: AION BIOSYSTEMS, INC., Darien, CT (US)

(72) Inventors: Thomas Blackadar, Auburn, NH (US); Peter Costantino, Westport, CT (US); Kristen Warren, Waltham, MA (US); Samuel N. Cheuvront, Franklin, MA (US); David Goodall, Chelmsford, MA (US); D. Alexander Colburn, Lowell, MA (US)

(21) Appl. No.: 18/678,366

(22) Filed: May 30, 2024

Related U.S. Application Data
(60) Provisional application No. 63/505,427, filed on May 31, 2023.

(51) Int. Cl. A61B 5/01; A61B 5/00

(52) U.S. Cl. CPC A61B 5/01; A61B 5/6823; A61B 5/6832; A61B 2560/0214; A61B 2560/0223; A61B 2560/04; A61B 2560/045; A61B 2560/0462; A61B 2562/0271

(57) ABSTRACT

The present invention relates to a system and method for, for example, periodically collecting physiological parameters in real-time from a plurality of subjects, for example temperature from cancer patients, heartbeat rates from persons being treated for coronary conditions, physiological orientation information mechanical shock and related parameters, for example from football players in danger of head trauma, and other physiological measurements which it real-time convey useful information. This information is coupled to a system which integrates the information, subjects it to criteria (for example doctor-specified dangerous condition criteria), communicates information and, optionally provides alarms to clinicians. The invention, through the deployment of computing functions on smart phones connected to the skin applied transducer collecting physiological parameters reduces power consumption, and this objective is further achieved through the use of sampling strategies and timing, and the limitation of access to the skin applied transducer.

### TECHNICAL FIELD

The present invention relates to non-invasive, wearable, and portable medical devices, methods, systems and apparatus for monitoring physiological parameters allowing both interface with information technology infrastructure and direct to human information communication.

### CROSS REFERENCE TO RELATED APPLICATIONS

This application claims the benefit of Provisional Patent Application No. 63/505,427, filed May 31, 2023, and entitled Secure Systems for Measuring Physiological Parameters, the disclosure of which is hereby incorporated herein by reference. This application also incorporates by reference the United States Patent Application No. ______ entitled Skin Applied Sensor System for Monitoring Human Physiological Parameters filed on or about the day on which the present application is being filed.

### BACKGROUND

There are presently a variety of personal health monitoring devices and systems on the market driven in some part by recent advances of sensor, electronic device and power supply miniaturization.

Small body worn devices that monitor one or more vital signs and transmit the readings wirelessly to a receiver unit without encumbering a patient with cables. A short-range wireless connection such as Bluetooth™ may be used. Typically the wireless receiver unit retransmits the data to an IT system for processing and display. The data (raw and/or processed) may be stored in a database or electronic medical record. Within the IT system or individual patient record various “rules” may operate to alert medical staff when one or more of the vital signs moves outside the limits set for a patient.

It is also known to provide wireless heart rate monitors which are held against the patient by a chest strap. Using a chest strap for support means that it can be possible to mount a larger wireless transceiver including a data processor that is able to process and/or transmit instantaneous data relating to cardiac function. However, not only do such devices tend to be heavy and bulky but the degree of contact with the skin is generally poor and prone to motion artifacts. Moreover, the need for a chest strap to be fitted around the subject means that such monitors may not be suitable in trauma situations where the subject is physically injured or disabled.

Monitoring devices that combine heart rate measurement with other vital signs, such as temperature and respiration rate, generally utilize separate sensors for each parameter and read each sensor sequentially. In addition to failing to provide continuous readings, such devices are also unable to provide concurrent data, for example simultaneous heart rate and respiration rate measurements.

Fitbit Ultra is about 2 inches long, 0.75 inch wide and 0.5 inch of depth biometric monitoring arrangement It has a pixelated display, battery, sensor, wireless communication ability, power supply and interface button of the encapsulation in this small volume, And the integrated intermediate plate of the other parts for attaching the device to pocket or clothes.

Apple's recent Apple Watch offerings includes health monitoring functions. However, Apple states that the “temperature sensing feature is not a medical device and not intended for use in medical diagnosis, treatment, or for any other medical purpose. Apple further states that “The Cycle Tracking app should not be used as a form of birth control. Data from the Cycle Tracking app should not be used to diagnose a health condition”

Temperature measurement is a key measure for infectious diseases as the onset of infection from compromised immune systems, as in oncology patients, the flu, for other viruses such as Covid 19. Treating an infectious disease early reduces hospital admissions, and in particular to which manner of care the patient receives, (patient room, vs. critical care vs. full isolation). There is also an unmet need for hospitals to remote monitor patients who are post chemotherapy and post-surgery to reduce the potential risk of suffering from sepsis.

It has been proposed to perform full data processing on a wireless sensor node rather than transmitting raw data. It has been reported, however, that in previous attempts the increased power consumption from local processing counteracts the limited savings in the radio power from a reduced rate of data streaming. Thus, there remains a need for an effective system for local signal processing and data transmission while having accurate, dependable readings.

### SUMMARY OF INVENTION

The invention relates to a cutaneous information device (“CID”), a skin worn devices for purpose of data collection, electronic systems integration, physiologic and other skin applied sensing and delivery of dynamic, unique, authenticated, and secure content allowing both interface with information technology infrastructure and direct to human information communication in a structure which provides independent and redundant human and machine-readable information.

A multi-parameter monitoring system that includes a cutaneous biometric information measuring device to measure with an extended battery life, an associated data capture device, a post-capture data processing for temperature data extraction, configurable system architecture, compatibility with additional devices like a blood pressure cuff or a digital weight scale, the ability to modify the sensor data processing parameters in the field without hardware modification, and secure data transmission. In preferred embodiments, the system would also have the capability of the system to “backfill” any data gaps in the sensor real time data feed by using the corresponding bulk data transfer information.

In a preferred embodiment, the system would include at least one CID, more specifically, a skin-applied biometric factor measuring device, comprising: (i) a flexible adhesive member for secure attachment to the skin; (ii) a biometric sensor integrated secured by the flexible adhesive member for accurate biometric measurements; (iii) a microcontroller for processing biometric data from the biometric sensor; (iv) a wireless communication means for transmitting biometric data to an external device; (v) a power management system for efficient power utilization; and (vi) a non-rechargeable battery providing power to the device, wherein the device operates continuously for an extended period without requiring recharging. The CID would be designed in conjunction with and paired to the data capture device referred to here as the data fusion aggregator (DFA). The DFA would be communicatively coupled to the CID monitoring device, configured to receive, and store biometric data transmitted from the monitoring device. The DFA would also convert the data from sensor(s) into useable data by analyzing, and processing the data for further analysis or display. The system requires that the DFA and the CID are tightly coupled together for proper data management and power control. The DFA and the CID define the operation of a single sensor. The DFA establishes the security protocol for the CID, defines any CID specific configurations, such as Time of Day, data sequences, data transmission frequency, and encryption techniques. If there are CID specific tasks that can be setup, the DFA can set these up. The CID ultimately controls the power consumption of the sensor device and has guard-rails that prevent the DFA from consuming more power than was agreed upon in the design of the DFA-CID system. The configurable system architecture could work with a single device, where biometric measure such as and the data capture device are integrated into a single unit. The system could also work with multiple devices operating in parallel, wherein multiple skin-applied biometric factor monitoring devices and data capture devices work simultaneously to monitor and capture biometric factor data. Each sensor in the system will have a unique DFA to collect and process data as agreed upon by the two sensors. There may also be used a hierarchical system, wherein a central data capture device receives data from multiple DFA-skin-applied biometric factor monitoring devices, to measure any number of factors, impact, concussion, or movement related data, and performs data aggregation and analysis at different levels. In some preferred embodiments, there would be an interface for accommodating additional devices, such as a blood pressure cuff or a digital weight scale, enabling simultaneous monitoring of temperature, blood pressure, and weight. The system architecture would require a DFA to be configured for each such device. The DFA can comprise an adjustment or update mechanism allowing the modification, change, or refinement of the parameters for processing biometric sensor data within the skin-applied biometric factor monitoring device, without requiring CID hardware modification or replacement. These updates could be manual and customizable or automated and triggered under certain conditions. In a preferred embodiment, there is provided a mechanism by which sensor data is transmitted in real time at a prescribed intervale and periodically forwarded in bulk form such that the bulk data can be used to “backfill” gaps in the streamed data due to the CID being out of range of the DFA. The system would comprise secure data transmission protocols ensuring the integrity and authenticity. While the system may be used to measure a number of biometric factors, the most prolific and useful data point across many applications, is the measurement of temperature. The System consists of a master server that is configured with individual parameters of the subject device to provide data. Such information could be physiological, demographic, environmental, location or other such information. The master server would provide the core system with the appropriate information including the unique identification (serial number) of the CID being allocated to the configuration set forth by the central server. This information is then made available to the DFA at the point of setup for the device. This establishes the communication path of the data from the DFA to the master server.

For example, temperature measurement is a key measure for infectious diseases as the onset of infection from compromised immune systems, including oncology patients, the flu, for other viruses such as Covid 19. Treating an infectious disease early reduces hospital admissions, and in particular to which manner of care the patient receives (patient room, vs. critical care vs. full isolation).

In accordance with the invention, the system comprises a secure cutaneous information device (CID) comprising at least one high-precision and high-accuracy sensor. In preferred embodiments, the CID comprises a device that is worn on the chest (or other locations on the body), and in combination with the associated system as the capability of monitoring body temperature within an accuracy of +/−. 1° C. and when used with typical application software can maintain its functionality for a period of, for example, over sixty days. This time line for length of functionality can be extended, with larger batteries, at a cost of weight and size (and functionality)

The inventive highly precise chest worn CID addresses a basic substantially unmet or poorly met needs for outpatient monitoring. In accordance with the invention, substantial life in a lightweight wearable sensor can be achieved using a very small primary non-rechargeable cell as a power source as appears more fully below.

While rechargeable cells may be used within the device of the present invention, the recharging of a remote sensing device poses additional challenges. For example, essentially, during recharge, the unit is out of service. This is because, during the recharge period, the user is not able to wear the device. The user may also forget to put the device back on.

In wearable sensors there are two functions that consume the most power, namely the power consumed by the sensor measurement, and associated signal processing; and the transmission of data off body, for example the communications link between the sensor and a smart phone being carried by the patient. The inventive system manages both the incoming and outgoing communications at the DFA-CID interface (for example by software located on a smartphone carried by the patient, and/or in the cloud to which the CID is connected by the patient's smart phone), as well as both the incoming and outgoing communications at the CIT. The CID sensor uses a high speed two wire I2C temperature sensor that is ultra-low power, such as, for example the Analog Devices Inc. Maxim Integrated MAX31855RASA+T.

Because body temperature does not change rapidly in normal daily life, in accordance with the invention, the CID sensor can be set to a relatively slow predetermined default data read rate, i.e. the time between successive temperature readings. This may be determined taking into account the application, patient age, patient lifestyle and so forth, as well as the desired period of time over which monitoring is to be performed. Based upon this, battery size and device communications range may be integrated into the design balancing the design to achieve acceptable parameters for weight, range and battery life.

The temperature is put into a broadcast beacon, and encrypted such that only a receiving DFA that has been authenticated can receive the broadcast beacon. Should environmental changes such as increased ambient temperature, the DFA can request more frequent next data measurements from the CID in order to protect a firefighter, an immigrant detainee, an athlete or soldier. This is done with the understanding that the power may be compromised. The architecture of the system does not require the CID to turn on the receiver and listen for the DFA. This method saves substantial power, and puts the burden on the DFA to perform the additional communications with the CIT. The DFA is unique to each CID and as such, with permission of the CID the DFA can set basic parameters in guidance with the power requirements of the CID and the data requirements of the DFA. The DFA does the management of all data received from the CID, and determines when there is missing data, the urgency of needing to obtain data, and at predetermined thresholds will break from the default communication policy to require the CID to stream data to the DFA.

In preferred embodiments, the DFA is housed in a local device (such as a smart phone) within typical Bluetooth or other personal networking range. A key component in this system is the unique protocol between the sensor and the DFA. There are key elements of the CID module such as data transmission rates, maximum time of data transfers and the DFA communication and data collection is designed with each sub-component (CID) connected to the DFA. To be explicit, this enables the CID to do the minimum amount of processing, and the minimum amount of communication with the DFA. The DFA is part of the CID system design that enables accurate physiological and environmental calculations, while ensuring the lowest power and lowest cost sensor for the system.

In principle, it is possible that the DFA be a virtual DFA located in the cloud, for example a logic circuit having a single input for recognizing DFA inputs from multiple sensors and persons, and then routing the sensor (e.g. temperature, blood pressure, movement detectors) to an assigned DFA subroutine.

In preferred embodiments, the system can be used with outpatient and inpatient hospital or medical facility populations. In a preferred embodiment the system will measure temperature, with the CID comprising a least one sensor on or near the chest and will transmit signals that can be converted to skin temperature via a Bluetooth connection to a mobile device, or other gateway device. Once signals are received, the DFA in the receiving device will calculate core body temperature, as well as environmental and physiological conditions to predict the actual core body temperature. In preferred embodiments, the DFA is connected with cloud services provided by the operator of the inventive system at the system operator's server in the cloud, the resultant data is transmitted to a clinician portal for next steps.

At this point the DFA can hand off to the cloud services, sending the computed data for further processing at the system operator's server using machine learning and other AI techniques such as random forest etc. Recognizing that battery size and thus device's active life can often pose a significant limitation to skin applied devices, the system, as will be described below, places reduced power demands on the CID itself by reducing the number of two-way data connections between the DFA and CID, optimizing by integrating at the time of design functionality, data paths and shared processing requirements ensures the low power, highly accurate sensor design.

In a medical setting, the system may also be used to 1) verify the identity of the patient with a CID by allowing the care giver to have an authenticated DFA, or by scanning the near field radio chip on the CID, 2) track and analyze vital signs indicative of the success of patient care or generate communications to healthcare professionals and/or databases. In some embodiments, an optically readable code, or NFC memory may be incorporated into the inventive CID for scanning or an extra layer of security, compared to other methods of identifying the patient (for example a facial recognition sequence initiated by a healthcare professional) and associating the readings from the device with the patient. In other embodiments, in addition to or instead of facial recognition, confirmation of identity may be performed through other biometric measurements. In accordance with the invention, it is contemplated that the same may be in addition to the signature of a noncontact communication device embedded in the CID.

The system is advantageous as compared to typical prior art personal monitoring system as it is mechanically configured so that it cannot be separated from the person once authenticated, for example, the system may be designed in such a manner that it is secured to the skin of the user by an adhesive layer that can be relatively easily separated from the CID in the event of the application of sufficient force to the CID, leaving the adhesive layer on the skin but with its adhesive characteristic substantially compromised or nonexistent. Security can be assured through a number of means including sensors, markings, NFC tags, biorhythm profiles.

### BRIEF DESCRIPTION OF THE FIGURES

A more complete understanding of the present invention and the advantages thereof may be acquired by referring to the following description in consideration of the accompanying drawings, in which like reference numbers indicate like features, and wherein:

FIG. 1a is a schematic overview of the system showing a personal Data Fusion Aggregator (DFA) in use with multiple CIDs;

FIG. 1b is a schematic overview of the system showing a personal Data Fusion Aggregator (DFA) in use with multiple CIDs organized in a hierarchical manner;

FIG. 2 is a schematic overview of the system showing a preferred embodiment of the Data Fusion Aggregator's functionality within the system of FIG. 1 with a single CID;

FIG. 3 is an expanded schematic overview of the multiple layers of a preferred embodiment of a CID to measure temperature;

FIG. 4 shows top and bottom views of the CID assembly of FIG. 3; and

FIG. 5 is a simplified schematic of exemplary circuitry used in connection with the CID of FIG. 3.

### DETAILED DESCRIPTION OF INVENTION

Generally, in accordance with the invention, methods for initial authentication and subsequent reauthentication has been described in our previous applications, which are referenced above and are hereby incorporated herein by reference. Activation of the device and associated system is initiated by a person providing identifying information to be associated with the CID in the associated inventive system. The information can be communicated to a central database/processor directly or via a local server/processor through, for example, a plurality of Internet connected computers, or, as illustrated, cellular smart devices (such as smartphones). Cellular smart devices are connected, via cell towers and cyberspace to a central server. Upon the initiation of communication with a particular CID with the central server, information is checked to determine whether the CID is already registered on the system or whether a new patient record needs to be created. After the patient is determined to have been registered on the system, or a new patient record created, the CID is applied to the surface of the patient's skin using an adhesive layer associated with the CID. The CID in connection with authorized devices can then be used to verify the identity of this individual.

Information collected by the CID of the patient is then stored on a central server database and may be further processed using data not available to the CID to enhance the accuracy and validity of the data. This is done using such AI techniques as and not limited to neural networks, Linear regression Generative AI models, associated the demographics in the patient record. The demographics to include physiological, age weight, height, medication, disease state and other information. This data predictive or analytical is passed to the patient record for the physical to analyze and prescribe further treatment. Access to the information contained on the central server will be managed by security protocols to ensure that the information being provided is on a need-to-know basis. While cloud connection is ideal, continuous cloud connection is not necessary for functionality. As an alternative to continuous connection, the device comprising the DFA, such as a As an alternative to continuous connection phone could be put into low power mode, where data would be stored locally on the device, and then uploaded in due course according to a DFA-CID pre-determined-protocol for communications. For use in a closed setting (e.g. hospital, rehab facility, cruise ship), local connection to a centralized server may be sufficient where the DFA-CID connection may be done via a gateway device, such as a smart phone which communicates with the CID and the local centralized server. The local centralized server would be equipped with the AI Module for additional process prior to passing CID sensor data to the persons record.

The Data Fusion Aggregator (DFA), in accordance with the invention, is implemented as software, for example, on a smart phone with which the CID is in direct communication. The architecture of the DFA comprises multiple logical units. These units enable accurate data computation from the raw sensor data in combination with preprocessed data. For example: The data coming from the sensor is skin temperature. The DFA is enabled with one of several algorithms; Machine learning, basic translation from skin to core, as well as AI techniques that would otherwise consume power in the CID. The DFA is to provide a mechanism for connecting for authorized sensors on the network and pass data to the cloud with the device signature. The DFA enables the sensors to perform their measurements. The DFA is part of the sensor sensing module CID. The DFA is configured to communicate with the sensor, do data management, as per the design of the sensor. This allows the sensor to save power in data processing, and management, and allows the sensor to send privately encrypted data without having to turn on the sensor receiver and wait for communications back. The communication and processing optimization is done on a sensor-by-sensor basis, and the DFA communicates and processes data and is, optionally but advantageously, unique for each type of CID (sensor) in the system. Each CID type has a unique data protocol, and access needs. For example, the data transmission needs and data payloads for a temperature sensor are much less than EKG and arrythmia detection. Thus, these are two different CIDs require two different DFAs for each CID to operate. The DFA manages the security, non-physical system frangibility and communication channel between the sensor and the cloud and processes only sensors that have been authorized to be on the network. The DFA identifies a CID but requires authentication from an upstream device/server to enable communications between the DFA and the CID. The DFA is the device that is authenticated to receive specific CID data. The data from each CID is time stamped and each DFA can report to an application that can then time-correlate the data. If appropriate the DFA send additional configuration information for a DFA to present to the CID.

The authorized DFA functionality can also accept settings from the central server that are applied to enhance the precision of the sensor functionality. Settings can be age, gender, frequency of measurements, weight, height to mention a few. Settings can include non-physiological conditions such as environmental conditions. This decision making happens above the DFA and CID, and is driven by the specific applications looking for specific results. For example, a sports or fitness application may have two sensors, one temperature CID the other a heart-rate CID, each with their own DFAs. The two DFAs communicate to an application upstream for the sensor system. The temperature DFA-CID capture core body temperature from normal living, the heart-rate DFI-CID captures heartrate. However, as literature has indicated (U.S. Pat. No. 10,702,165-B2 to Buller), when heart-rate reaches a certain level above everyday living for extended periods of time with appropriate algorithms the heart-rate is a better indicator of elevated temperatures of the core vs. skin-to core temperature derivation. At this point the upstream application can make the decision to use the heart-rate to core conversion until-such a time that the heart rate returns to normal. The heart-rate to core temperature conversion relies on knowing what the pre-activity core body temperature was, and the Temperature CID was providing that information. Therefore, a simple heart-rate monitor, and a skin-to-temperature monitor together become a high-performance sensor without having to sacrifice power, calculation to achieve the superior performance.

In preferred embodiments, the DFA module comprises a secure software package that has a simple API (application programming interface) such that it is easily integrated into smart devices with the appropriate communication paths, and processing power. The DFA resides in smart-watches with internet connectivity, mobile handsets, WIFI and cellular gateways, Personal computers, real-time processors and can be implemented in the cloud. The multiple DFAs can be integrated into a single wearable sensor to control multiple CIDs and time correlate data with a light-weight application. For example: a smart watch could have several DFAs and be able to control several CIDs.

A preferred sensor system with the DFA and desired sensor functionality are architected together to optimize lowest power sensor design, with the highest quality data being presented to the user, clinician, coach or parent. The specific parameters of the DFA can be tailored around the desired senor functionality. However, it is highly desirable that DFA processing be done in a nearby device to ensure accuracy and continuity and reliability and feedback. The DFA first receives a request from the application to connect to the CID. The DFA then queries the CID, in the preferred embodiment, via an NFC read command at 1 inch range from the CID. This provides several functions; a secure read of the identity of the CID (seral number) and the means of communicating with the CID (PIN Number); this will cause an interrupt to the microcontroller on the CID and take the CID out of storage mode and into operational mode. The DFA will queries the application, the application queries the central server for authentication purposes. The central server can send back a “not registered to person”, or “authorized to communicate”. With the authorization being positive, the DFA can then send configuration information such as and not limited to: Time of Day, measurement recording interval, communication frequency, and encryption techniques. Following that in the instance that Bluetooth communication is used, the DFA listens to the CID beacon for temperature readings and processes the data with a prescribed algorithm for converting the skin temperature to body temperature, and pass the data up-stream to the central server for further processing. The DFA anticipates so many readings per hour as defined by the recording interval. There will be missed readings and when a threshold of missed readings is missed, (this depends on battery size and CID design, the DFA will perform a communication bond with the CID and request the missing data from the data store. The CID is design with a maximum amount of data that can be broadcast at any one time based on the power source and the ability to provide the high current required and the time it takes to deliver the messages. In a preferred embodiment the CID is using a CR2016 battery (Eveready) that can send peek current for 10 milliseconds. Thus the data packages are limited to that. As batteries age during continuous near the end of life of the battery the CID may limit the amount of time that data can be transmitted to extend the life of the CID. The DFA is designed such that it is aware of the CID's requirements for battery preservation. The CID at the point of wakeup, performs a system diagnostic, initializes memory, computes the unique pin, checks the viability of the sensors on the I2C and sets time of day, and provides manufacturing information in accordance with the ISO 13485 quality requirements for certification of the device. The CID then sets internal timers on the 32 kHz timer and turns off the 32 mHz clock and goes into a sleep mode to conserve power. When the measurement frequency timer reaches 0 the CID will wake up and process the temperature sensor data, store the data and the sequence number flash, and the advertising beacon, then return to the sleep mode. Should the DFA determine that the threshold of missed data is reached, the DFA will send a read request to the CID and that will wake up the CID from sleep and the DFA-CID will bond and share the data from the storage. When complete or if the CID breaks off communication because the length of the communication is too long, the CID will set the 32 kHz timers and shut off the 32 mHz clock and return to sleep mode.

Sensor placement on the body or in home varies depending on the functionality required. Thus, in some instances one may have multiple sensors on the body and want to combine sensor input to enable a higher level of diagnosis. Some sensors are not on the body and that data may want to be combined with a patient, such as room temperature from a smart thermostat, the weight scale in the bathroom, blood pressure monitor, pulse oximetry, blood-glucose, sleep sensors in a mattress etc. All these sensors can be authorized as belonging to a single or multiple individuals through a higher-level authorization module that can be communicated to the DFA. For example, a central server (which is capable in accordance with the preferred embodiment of servicing a number of individuals) may be configured to have five devices assigned to an individual. Each Device has a DFA unit connected to a CID. These are configured to be securely identified to a single user. When the user connects the devices to his/her person the server application authenticates the CID sensor and DFA as belonging to the person. This association can be done via a near-field-communication radio (4 inch transmission distance) and the device is securely connected to the server. Thus all the sensors in the system may have NFC identifiers and one system with multiple DFA and CIDs can be registered to one unique individual. The application that sits above the DFA and CIDs, and coordinate the data, as in the to make a more valuable data set. This can be done in multiple situations. With an activity sensor collecting stride information and combining it with the inter-beat-interval will provide a fitness index. The design of two CIDs is simple and, in both instances, requires very little power. Combining a heart-rate sensor and a stride sensor and time-correlating information for both will cause the stride sensor to consume more power as the EKG sensor or SpO2 sensor will consume more power than the stride sensor and the combined sensor will consume more power thus requiring larger size and weight. In the case where two temperature CIDs are used each will have its own DFA and at the time of configuration the body location will be identified to the DFA, and the DFA will be configured to pass raw data upstream to the data processing engine for ML or AI techniques to determine deep vein thrombosis on the patient.

The preferred system's the temperature CID comprises a near field communications chip ST250DV Micro with memory storage. This implementation of NFC provides for an open drain circuit to be energized and will generate an interrupt to the microcontroller a STMicro BlueNRG processor. The CID has two crystals, a 32 kHz watch crystal, and a 32 mHz crystal for providing the high speed clock for the microcontroller (these are generic crystals from multiple manufacturers. The microprocessor communicates the onboard temperature sensors. The CID has two of the temperature sensors and communicate over the two wire industry standard I2C bus. There are two sensors on the board, one facing away from the body, and one facing into the body. It is critical that there is no separation from the sensor to the skin of the body. The microcontroller has a fully functional certified Bluetooth radio. There are two antenna circuits that are etched onto the circuit card. An outer ring antenna for the NFC radio, and an inner antenna for the Bluetooth radio both with industry standard impedance matching circuits. Thus the construction of the sensor is important to the accuracy of the device. The skin side temperature sensor must have minimal gaps between the onboard sensor and the skin. To assist with this, the bottom surface sealing label is a 1 mm PET adhesive backed adhered to the body with a 3M-9917 Pressure Sensitive Adhesive (PSA) or equivalent. To further improve the conductivity a thermal brass stud is placed over the sensor and thermal past is applied to fill the surrounding cavity displacing air. To further improve the thermal properties of the temperature sensor there is a neoprene layer adhered to the skin side of the circuit board to further remove air from the bottom side. This is adhered with the same adhesive. On the ambient side of the circuit board above the ambient temperature sensor is also a thermal stud that protrudes up through the frame to be as touch the final sealing label a 1 mm branded (or not) PET layer with the same sealing adhesive on the bottom. The reason for the top layer it to provide additional data for upstream processing and to provide a thermal gradient. The entire enclosure is waterproof by construction. There is no skin facing adhesive on the bottom side of enclosure as this will present thermal resistance between the skin and the sensor, as well as limit the wearable life of the sensor. Instead to make the sensor more comfortable we use a silicone adhesive from Medway Inc, called the Medvance silicone tape that is applied over the sensor and to the skin. This embodiment uses 2 inches by 3 inches of silicone tape for secure adhesion to the skin.

The system's CID comprises a near field or other communication protocols (Bluetooth, RFID, etc.). In preferred embodiments, the system would comprise a skin wearable, waterproof, device comprising an adhesive, sensor, battery, Bluetooth, in combination with at least one reader device with a Bluetooth enabled DFA interface device able to receive information from said CID to process the sensor data and from said reader device respecting the individual identified by said individual identification device; a computer system coupled to said computer interface device, said computer system including a memory and means for processing information collected by said computer system.

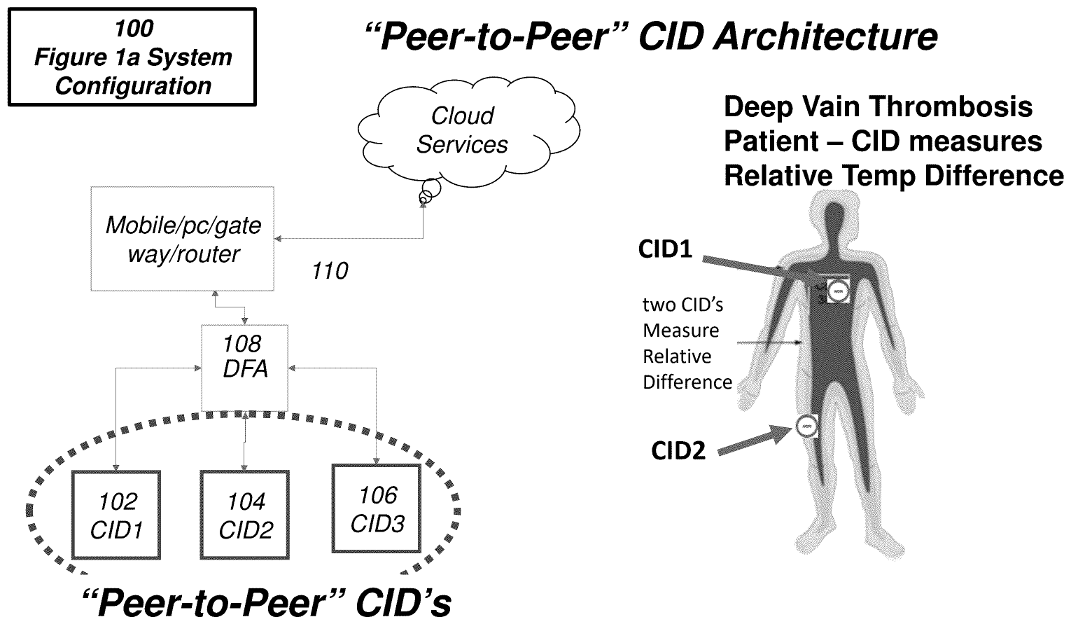

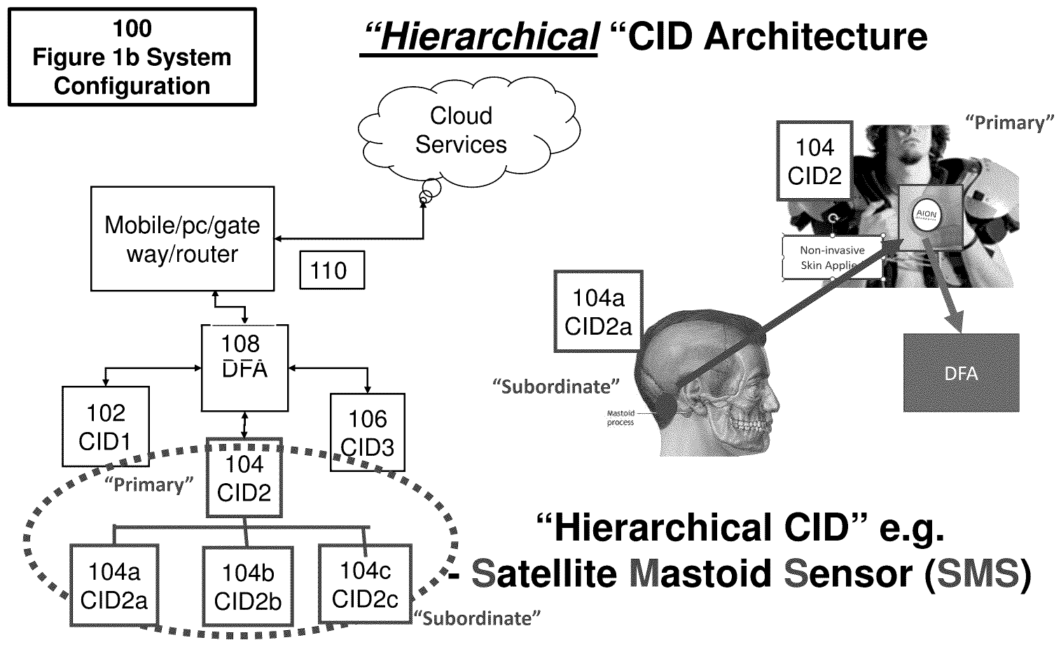

FIGS. 1a and 1b are schematic overviews of the system 100 comprising multiple CIDs 102, 104, 106 that are connected each to a data fusion aggregator (DFA) 108,111 and 112 which can be housed in a smart watch, laptop, mobile device, gateway set top box. DFA 108 would have the capability to connect to multiple CIDS, as well as a server connection point 110, which could connect to cloud services which could be remote or within a localized network (e.g. hospital, cruise ship). One or more CIDs with dedicated DFAs can be used to measure various biometric data including temperature, heart rate, blood pressure, and other data points such as respiration, body positioning etc. Alternatively, CIDs 102, 104, 106 are worn on different persons and the associated DFAs in 108,111 and 112 connects with an authentication database that authenticates the respective identities with the respective CID-DFAs. For example, a deep vein thrombosis patient may wear two CIDs to measure the temperature difference between the locations. In an alternative embodiment, FIG. 1b shows a system the CID-DFAs push the data to the app as described in detail below. When the CIDs are configured in the system it is known from the system configuration that the CIDs from their respective DFAs are connected to separate limbs on the body, and the application provides guidance as to which leg each CID is on. The DFAs keep the body-registration information leaving the CID in an operational state that is the same as if it were placed on the chest to achieve core body temperature. The DFA-CID coordinated by the Application will receive skin temperature data and will not convert this temperature to core-but pass the skin temperature to the application where the application will discern if the two sensors have the significant temperature change differential to indicate an issue with the patient.

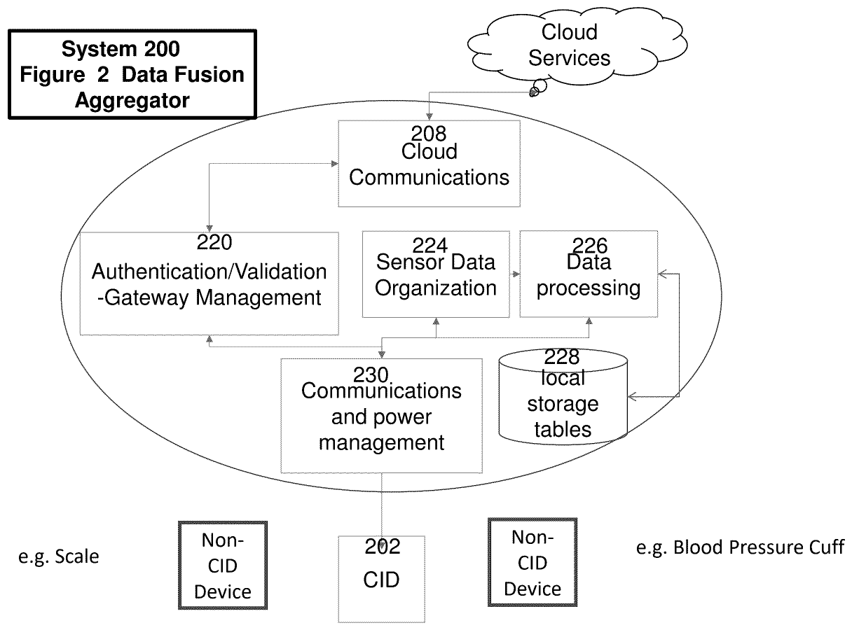

FIG. 2 shows a preferred embodiment of a system 200 with a singular CID 202 to accurately measure temperature. CID 202 relates to DFA 208. DFA 208 is comprised many functional modules 220 including cloud communications 210, activation/validation management 220, sensor data driver 224, data CID specific calculations 226 which will process data and then check processed data with local storage tables 228 (outside the DFA). Finally, there is communications and power management module 230 which can turn CID 202 on and off to manage power and keep track of the offloading of data and set up the communication schedule with the sensor. The controlling system, mobile device or computer or gateway can also be connected to devices such as a blood pressure cuff 232 or a weight scale 234. Though no CID devices in the system there will be a DFA to harmonize the system architecture and manage these devices.

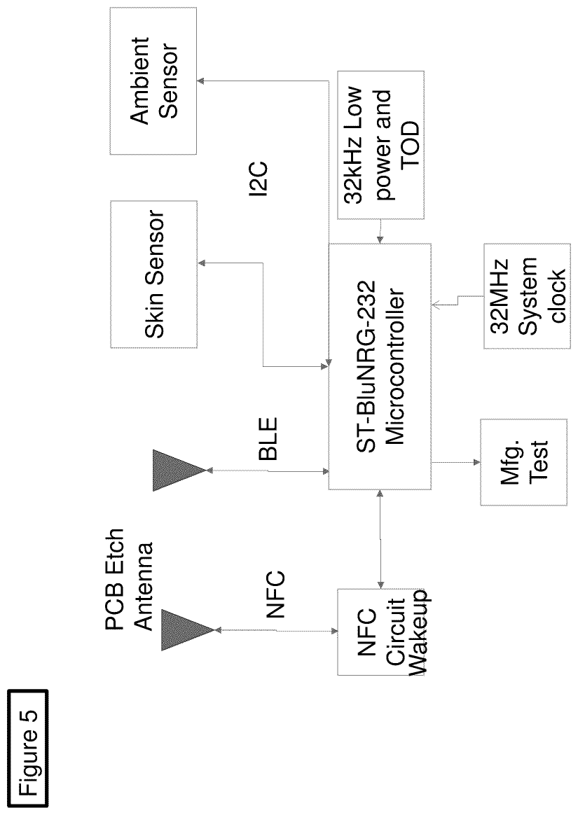

In FIG. 2 CID 202 has a unique registration number. The CID 202 as configured and later shown in FIG. 5, has a factory programmed NFC Chip with a wake-up circuit that will broadcast a near field RFID unique ID for the chip. This uniquely identifies the CID 202. This unique number must be available to the local cloud communications block 222, that connects to the DFA, when the Local cloud communications block requests communication of unique address of CID 202, if the CID 202 unique address agrees with the authenticated users unique address of 202 DFA can communicate with CID 202. Alternatively, the mobile device authenticates the user to a cloud service that has pre-registered CIDs and if the CID 202 is free to use is discovered using 222 the 208 can authenticate the 202 communications. As referenced above CID 202 is configured to communicate sensor information in a transmission only beacon. The CID 202 has been designed for size and weight and longevity of battery life. Thus, radio information required to do further configuration or to retrieved missing data is limited by design of the 208 and the CID 202. The CID 202 can refuse requests for connection to the 230, and the 230 can use priority requests, that the CID 202 can still refuse. If the CID 202 chooses to permit two-way connection it can limit the amount of time due to battery characteristics. Data processing in 226 is designed to minimize the CID 202 radio traffic.

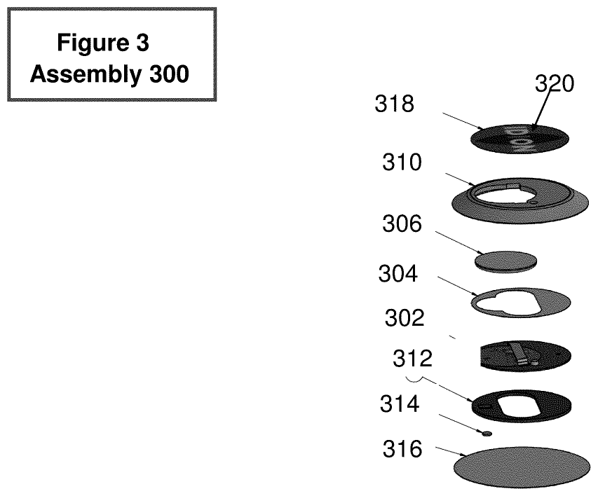

Referring to FIG. 3 is an expanded view of a preferred embodiment of CID 300 that could be used in connection with the system of FIG. 2. CID 300 comprises Sensor PCB (printed circuit board) assembly 302 which is connected to PCB pressure sensitive adhesive 304, where battery 306 is installed.

Assembly 308 is inserted into the frame 310 of the sensor module 300. On the skin side of the PCB part is the skin side temperature sensor (Tsk). Mounted on top of is a uniquely designed heat pipe which is placed against the sensor and the air gap between the inner seal is filled with thermal paste. More specifically, Foam filler 312 is placed on the bottom of the circuit card to fill any air gaps and then skin temperature sensor heat pipe 314 is inserted into the opening and any remaining air gaps are filled with thermal paste (not shown). Then the PET inner seal assembly 316 is applied with foam filler 312 and sealed to frame 310. Final assembly to ensure waterproofness includes a PET cover with 318 with a marking, optionally a covering with printed information 320. Battery 306 is connected to frame 310 connected to an inner seal assembly 316 connected to foam filler 312 connected to the PCB (printed circuit board) 302 with PSA (pressure sensitive adhesive) 304 and thermal paste.

The construction is waterproof to IP 57 per IEC 60529. This marking could contain individual specific information which can be visually seen, read or scanned for patient identification, interaction, information exchange, and instructions and a non-contact communication device. The construction is waterproof to IP 57 per IEC 60529, as noted above.

In a preferred embodiment, inventive CID 300 comprises a transducer, a programmable DSP and battery 306. The local device, such as a smart phone, in communication with CID 300 comprises communications interfaces for sending and receiving information to and from CID 300, as well as a digital signal processor, such as a CPU, for processing information received from CID 300 and communicating it to a central server. When considering the operation of the local device such as a smart phone, The operation of the communications interfaces for sending and receiving information to and from CID 300, as well as of the digital signal processor, such as a CPU, for processing information received from CID 300 and communicating it to a central server begins and in communication, through the Internet, with the server operated by the operator of the inventive system.

The server located in the cloud, in communication with the local device, such as a smart phone, which is in communication with CID at present state we have a new president for your president you are present in for you 300 comprises communications interfaces for sending and receiving information to and from the local device, such as a smart phone, as well as a central processing unit, for processing information received from CID 300 and communicated to the central server by the local device.

When operating using a local device such as a smart phone, the operation of the communications interfaces for sending and receiving information to and from CID 300, as well as of the digital signal processor, such as a CPU, for processing information received from CID 300 communicates information to a central server through the Internet, with the server operated by the operator of the inventive system.

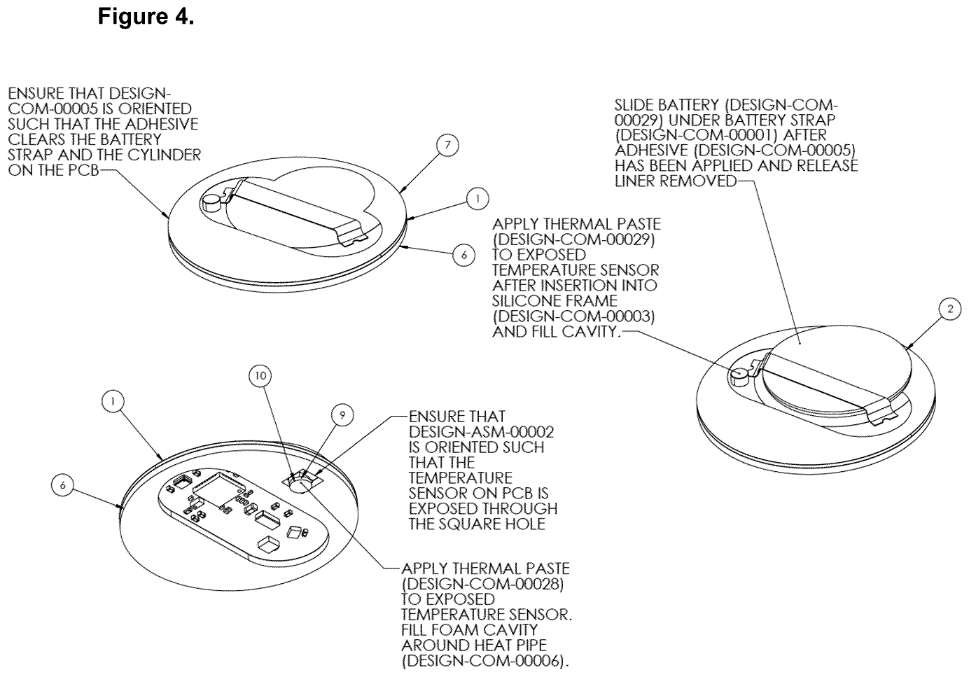

FIG. 4 shows a top view of assembly showing a thermal stud, battery clip as well as a bottom view of assembly which show the thermal stud, thermal paste and microcontroller, 32 MHz clock and a 32 KHz clock, The circuit card has two designed-in antennas: NFC chip and one for the Bluetooth Antenna-Bluetooth matching inductor (balun). To remove any air gap there is thermal paste inserted just above between the stud and the outer layer. Alternatively an assembly can be made of ABS plastic, screws, O-rings, to achieve the same characteristics needed to make proper temperature measurements and maintain the proper comfort level.

In preferred embodiments, both have RF matching circuits for proper operation and maximum efficiency. Under one thermal stud is the ambient temp sensor (not shown) and under the skin temperature sensor heat pipe the skin temperature sensor (not shown).

FIG. 5 shows a simplified schematic of exemplary circuitry used in connection with the inventive system. In a preferred embodiment, clock circuits are important components. There are two sources of clocks. The 32.768 crystal selected is the “watch” crystal for the CID. The 32 MHz crystal provides the processor high speed run-clock. The NFC circuit provides for identification and pairing of the device. The RFID designed antenna captures the energy of the 13.56 MHz ISO 14443 radio frequency (NFC band) and provides energy that powers up the NFC Forum Type 4 Tag IC (FL1). When energized, the NFC protocol allows for reading and writing the memory in the chip. The microcontroller (micro) has a fully certified Bluetooth stack. The 32 Mhz clock can be disabled and only the 32 kHz clock is running. The micro timing circuits will turn on the 32 Mhz clock and wake up as needed.

In accordance with the invention, it is contemplated that different sized CIDs may be used for larger and smaller people as well as varying application locations for the CID based on the environment. In a preferred embodiment, the device is less than about 6 grams no greater than 10 gm, 40-50 mm diameters, preferably less than 44 mm in diameter and all materials comply with ISO-10993 biocompatibility. Its small size can present thermal challenges for the integrated temperature sensors on the circuit card. The PC circuit card has a thermal stud that protrudes through the silicon outer layer and rests against the outer layer PET decal and all air gaps filled with thermal paste.

The CID is constructed to be optimized for size, weight, power and flexibility as a skin applied device in accordance with the desired characteristics. The accuracy of temperature measurement is critical, and air gaps are typically problematic when trying to measure the precise skin temperature, and when present makes the production reliability (CID to CID variances) inconsistent. The components in the CID must also have the appropriate characteristics to handle moisture either from external sources or that are naturally present on the skin, such as sweat. The CID uses on the skin side a thermal stud and thermal paste to remove air and provide a thermal path to the skin-side-layer. To minimize the storage requirements, and the processing requirements only the measured temperature is transmitted to the DFA. The DFA will then perform the necessary calculations required to convert skin temperature to core-body-temperature.

System 200 preferably includes including CID, DFA and possible cloud services. First, the CID 202 is activated. In a preferred embodiment, it is activated by an NFC. Other activation options include non-biological vibration sequence. Once the CID is activated the CID starts the sensing (such as temperature) and continues that programmed state. The DFA can communicate with the sensor if the sensor serial number has been previously configured in the cloud, mobile device, or PC. The DFA will set the date and time, and possibly other user specific parameters (not needed for temperature) and then the DFA goes into listen only mode. Module 228 communicates with module 222 for authentication or validation. If previously authenticated then module 224 organizes the data then sends the organized data to module 226 which processes the data including checking the data against stored information in module 228. If authentication fails, then module 222 will pass all data to the cloud communications module 232 to look for authentication. It is understood that cloud communications can encompass connections not just via the internet but also any suitable local area networks. If authentication is not immediately successful, the system can hold the data for later authentication (e.g. phone battery or application has compromised functionality).

In alternative embodiments, there could also be sensors to measure for pulse oximetry, heart rate, respiration, respiration, stride biomechanics, fall detection, and body position. The device would also transmit a signal and the DFA would process this (e.g., RSSI signal) such that in a hospital equipped with Real Time Location Services (RTLS); the location of the patient can be achieved. The associated LTE gateways combined with DFA capability allows collecting data from additional devices (which may or may not be skin applied) such as weight scales, and blood pressure monitors, devices for EKG measurement specific to the patient, and can provide appropriate diagnosis at the point of reception. In accordance with the preferred embodiment, in each case there is one DFA for each sensor. Upstream of the sensor are application modules that can adjust DFA-CID-Sensor functionality as may be desired.

In accordance with an alternative embodiment, there may be provided a master DFA, which would have small data tables to improve data integrity. For example: The master DFA could be configured to collect multiple data streams from the DFAs associated with an individual and provide a synchronized time stamp for the data. The weight scale is in the bathroom, and there are multiple people with access to the weight scale. In determining the proximity of all the sensors belonging to the person one can determine the relationship to the DFAs of the sensors, and should the LTE or WIFI Gateway determine that the weight scale DFA and the person are in the near same location and the weight scale data is close/approximately the same as the last weight from the person the data can be identified as belonging to the individual.

In some embodiments the master DFA is configured to remove or tag artifacts of body worn sensors to avoid false positive indications of error. The master DFA can have a running list of measurements and delay a false sensor reading. These readings can be due to environmental changes, or due to motion artifacts.

The system is also different from traditional personal monitoring systems as while in use, Master DFA and each DFA-CID are bonded to the person's identity and cannot be separated from the person once authenticated and yet can still be used in connection with multiple local devices while the data processing is not done on the device but rather on the Master DFA or when appropriate the application designed for the specific application. In other embodiments, security can also be performed by doing a biological identification through the sensors by the Master DFA and the upstream application.

A hardware system constructed in accordance with the present invention and suitable for practicing the method of the present invention was illustrated in FIGS. 1 and 2 with a CID 202 paired with a DFA 208 as the nexus point of the system with servers and other devices communicating via DFA 208 to administer input and share critical information. CID 202 may interact with an authorized DFA 208. Authorized DFA 208 is a device that is used to interact with CID 202.

The essential components of the system comprise the CID and the DFA with the parameters disclosed above. In an alternative embodiment, the DFA would interact with cloud services and the CID and would comprise Bluetooth capability, local processor/database, which is linked via wired or wireless connection. In part of a larger monitoring system, there may be linkage to a central server. In an alternative embodiment, the interface directly connects to a central server. There is still optimized power management and reduced data transmissions by doing most of the work at the locally housed DFA (e.g. DFA is built into the gateway device and the central server). In this instance a component of the server too has the CID specific rules of communication and can command the gateway device to augment data collection if necessary.

Central server communicates with system inputs via the DFA. Central server may comprise a database with user CID design information generated by a user CID design. Such user CID design information is also stored in the database, which may be a hard drive, solid-state hard drive, or any other suitable storage medium, device, integrated sub system, and so forth.

Authorized DFA 208 also allows a connected device to push data to the central server for the presentation of data input screens, audio alarms, and the transmission of data to the central server. DFA 208 is an input and output device, and could interact with multiple entities. Similarly, a large number of DFAs unique to an individual could communicate with a central server. In the case of smart mobile devices, such functionality is typically incorporated though there can also be a separate stand-alone reader (e.g., a mobile scanner) for devices, which do not include this function.

The DFA can also be connected with a local device such as a medical facility glucose monitor, medical diagnostic equipment, radiology equipment and the like, or a remotely connected device (e.g. home medical diagnostic equipment); facility Y server (e.g. a server at a second facility), mobile device (e.g. smart device that is not tethered or bound by location or individual or equipment) all of which are connected via wired or wireless connection back to central server. Authorized DFA can be incorporated directly into these devices or can be an external stand-alone reader. Each of these devices from third party device manufacturers can be equipped with NFC tags that are registered with the central server to uniquely identify the device to the patient at the time of first use of the devices.

If an individual is taken to a care facility such as a hospital emergency room because of the incident, information from the system will be sent to these locations prior to the user arriving automating the intake process. During admission, hospital staff scan the user's CID using the application to view the user's health proxy information in real-time. If the user does not speak English, hospital staff can click the translate button in the CID application to use the voice and text translator to communicate with the user.

Additionally, users can scan their CID with their mobile phone to access their user portal and view certain information drawn from their admission and medical records. After discharge, users can scan their CID with their mobile phone to access their discharge information and view their ER diagnosis, expected course of illness, self-care instructions and return precautions.

In preferred embodiments, the CID is stiff enough to maintain its architecture and would not become deformed, wrinkled, and potentially not applicable to the surface of the skin. Referring to FIG. 3, print surface 318 is a flexible, breathable, comfortable printable material. The printed surface layer's top surface is intended to be printed on and display the contents of the print cleanly and clearly. The printable layer can include letters, numbers, designs, QR, barcode, data matrix codes and/or similar technologies for coding printable designs. If the printable layer is not practical for a specific CID, the CID can be scanned using the application via an NFC direct read. In a preferred embodiment, the CID would be effectively tactically imperceptible so that it would not bother the wearer. The individual could be identified using The CID embedded with a two-dimensional code such as a QR or data matrix code, other optical code system and/or an NFC RFID chip or other non-contact communication device. The individual can also be identified by the unique identification of the CID itself.

In a preferred embodiments the CID has no adhesive associated with it, and a simple off the shelf bandage can be applied over the CID to adhere the CID to the body. This provides the use the ability to change adhesives to reduce the risk of MARSI and skin irritation. In some preferred embodiments there would be a stronger adhesive on the edge meaning the adhesive on the outer layer will have stronger properties than the inner area, keeping the bond of the CID stronger at the edges, at the tension areas. This is also a way to reinforce the edges of the CID to prevent curling/peeling up. In an alternative embodiment, the CID may have two layers of adhesives-one that adheres the sensor on the body, a second layer of adhesive at the point of care can be printed on with the patient specific information and is applied over the first layer as part of the admission process for visual identification. While adhesives are described here, it is understood that alternative means of securing the CID to the user may be used so that the sensor is able to collect accurate information

These devices can also contain a security key code which would increase the security and functionality of the device by creating a counter within the chip that will prevent anyone from accessing or using the chip without the proper security key.

In some embodiments, there will be at least two sensors, where one is used for measuring a vital sign (e.g., temperature) to monitor the patient and a second sensor would measure another vital sign to sense tampering, removal, or malfunction of the device so that a tag could be sent to the central server to send an alarm to the system and to users or providers. In some embodiments, the device could also be deactivated if a sensor detects the subject/patient is not the same as the previous readings from the patient. Thus, the DFA can send warnings, deactivate, as necessary.

In a preferred embodiment, the device would comprise an RFID component (or other NFC device) having a signaling range 2-4 inches, preferably and a longer-range radio such as Bluetooth or single channel having a signaling range of 5-300 meters. In preferred embodiments, the device would be activated using NFC RFID. Using Bluetooth, the DFA-CID pairs to the phone or other smart device and transfers the unique ID of the electronics in the CID. In preferred embodiments, the device would have physical dimension ranges 22 mm-50 mm diameter×3.2 mm-6 mm high and a weight range of 3-15 grams. For uses where continuous monitoring is required, if the system detects tampering or malfunctioning of any sensors or other parts, the system may automatically deactivate the device and send an alarm. The system may also deactivate the device for any unauthorized use. By using Bluetooth, the DFA can identity of the patient can be verified and sensor data can be captured by an authorized device. The caregiver would not have to be in close contact with the patient. This is particularly useful with physiological sensors that are placed on the body to capture physiological data and are typically under the clothing. Additionally, it would be very useful in quarantine situations where vital signs can be taken from a safe distance.

Additionally, in alternative embodiments, the device can send out a signal to allow passive reading from a distance. In this scenario an individual can enter a room and with an authenticated DFA with their device immediately notify the appropriate devices of the person's presence.

The CID can include power on and sensing techniques well known to those skilled in the art. Traditional sensing devices perform functions locally where raw data is captured, analyzed, and converted to a humanly understandable output, such functions draining the power system. This inevitably compromises battery life which makes the device impractical for continuous long-term use. The battery on the present CID is basically used to power the sensor and any communication channel. In contrast to prior art devices, the present system comprising at least one CID and at least one DFA, the system can take on the challenge of taking minimally collected raw data and performing the secondary functions in the DFA or in the upstream application. This architecture allows for the CID to embed more sensors with less of a power demand with the CID having continuous use for at least 30 days in contrast to a device such as a smart watch that typically needs daily removal and charging. Each sensor in a multi-sensor device will have its own DFA. Thus if there are three embedded sensors in one device, temperature, EKG, and motion, there would be three DFAs associated with this one device that would manage each of these sensors.

Chest worn sensors for actigraphy and other uses are well documented and there are sensors in the field that perform activity measurements such as fall detection, step count, calorie count, body position and distance traveled. However, the present inventive system has the raw data from the accelerometer being analyzed and processed in the DFA portion of the system, while the other DFAs monitor the other sensors-such as heart rate and with temperature to obtain a true physiological profile of the subject under stress. This removes the power burden on the sensor to do extra processing and communications, or to combine and time synchronize the data from other sources. By moving these complex functions off the multi-functional sensor, the system allows for simple well-designed sensors to become accurate medical devices.

The continuously recorded raw data would be saved locally and then uploaded at convenient times as prescribed by the system architecture. The system would present to the DFA settings, and parameters from a hospital system where the clinician could prescribe parameters and boundaries for the interpretation of the data, frequency of data collected and radio transmissions. This could be done from the cloud directly. The use of the DFA-CID system, reduces latency and with proper rules in place allows for the lowest power communication and process of the sensor,

Thus, the system could be used to monitor for basic measurements that are relevant in any setting in or outside of healthcare and can be used as an early indication for medical conditions heat exhaustion, hypothermia, sepsis by measuring biometrics such as salt content, foreign compounds and even dehydration. For monitoring emergency situations (for example in a combat or bioterrorism zone) other sensors such as pressure sensors, radiation sensors, chemical weapons sensors, radiologic sensors, paper sensors may be useful. In some embodiments, the CID could monitor skin temperatures changes over time with the probability of developing hypothermia. Conversely, by measuring the changes to core temperature or hydration, the CID can be used as a monitor for heat exhaustion or UV exposure. The data could be saved on the DFA system and not locally on the system.

The sensor system is comprised of three components the sensor (CID) the DFA and the upstream processor. The DFA-CID definitions dictates all communications between the DFA and CID. There can be a hieratical DFA system where the Master DFA can communicate with the DFA-CID sensors that all are authenticated to the same individual in the system. The Master DFA coordinates the incoming data and presents it to the application as requested by the application. The resulting communications use the raw sensor information and applies novel methodologies, physiological, and environmental parameters. The DFA is the authentication element in the system coordinating authorized users and uniquely identifying the end user to the system. The DFA performs all specific data manipulations only for the CID that is authorized for said calculations. The DFA-CIDs that are authenticated in the system will have their data passed up to the central cloud databases, where further interpretation of the data can be done in a manner known to those skilled in the art. Thus any gateway device that does not have a display will only provide authentication services and any non-subject personalization. The DFA in a gateway instance is typically in the cloud and tied more closely with the upstream processor. The DFA presents to the gateway the metrics of the CID that is communicating to ensure the gateway does not make cause for undo data communication, while still enabling the CID with sensing parameter such as frequency of measurement and communications.

In one preferred embodiment, the CID providing data to the associated DFA, the DFA will execute the authentication process will authenticate the CID and the DFA as associated devices. Additional CID's may be authenticated as authorized in working with the cloud, the DFA will not do the further personalization process for the CID not authorized for the specific DFA. The DFA will further authenticate to the cloud, if successful CID data will be processed with the minimally approved data and then transmitted to the cloud services for further action. The skin temperature changes much faster than the core body temperature. These changes are easily detected and the DFA can be tuned to a body's circadian rhythms over time, and can be tuned to qualify both good and bad temperatures over short periods of time.

In a preferred embodiment a temperature senor will transmit raw data to and identification to the DFA. The DFA will authenticate the sensor. The DFA will process the skin temperature data to output a reliable core body temperature. The DFA can be given settings for alert temperature thresholds, physiological phenom including rules for what rapid skin temperature changes actually relate to core temp measurements, frequency of questionable measurements, and maximum latency to come to stable temperature readings. Should there be a need the DFA can instruct the temperature sensor to take more temperature readings per minute if there is a determined need (Firefighting, hazmat suits, athletes and soldiers). The DFA can completely turn off the sensor requiring an external means to turn on the sensor (NFC association). All of these instructions can come in the way of settings and can be prescribed by the clinician/trainer on an individual basis. Age, gender, height, weight are other such parameters can be input to the DFA for monitoring the associated CID. This personalized data is not pre-processed by the DFA for un-associated sensors and is left up to cloud services for processing (as this is the technique currently known to those skilled in the art).

In an alternative embodiment the DFA can be a sensor like device without sensors and applied to the body. Following similar construction techniques, or a package that can be affixed to a limb or torso with or without a strap. This DFA would replace a larger mobile device as the DFA and provide deeper analyze data and a master DFA or application can synthesize data from multiple (DFAs) sensors on the body. An example would be multiple accelerometers around the body to do analysis of data signals to determine impact, body movement, gate analysis, heart rate, and body-temperature.

The system enables a lower power load on the sensor, and additional bulk storage in the DFA, as well as a single communication path to the mobile offload or gateway. This will allow for multiple gateways that have only the responsibility for passing calculated data or semi-processed data to the server up stream.

In a preferred embodiment, the CID could be removed from the skin to replace the adhesive layer for longer use of the device. This would give an opportunity for direct visual examination of the area by the wearer or others, such as a medical professional. Security could be maintained with a two-factor authentication and by monitoring biometric data (e.g., signature of circadian rhythm, interbeat-intervals, gait analysis) to create a unique biometric signature to authenticate the single user, where any removal and attempted use of the device would fail. In a preferred embodiment, biometric factors can be used to confirm identity. To maintain security, there would be a pre-set time limit on the non-contact of the sensors to the skin. In preferred embodiments, the defined period of time would be set under 30 mins, preferably under 10-20 mins. Beyond that there would be an alarm sent to the system noting that the device would be deactivated without biometric reauthentication. In a preferred embodiment, biometric sampling (e.g., a combination of heart rate, temperature, pulse oximetry) would occur every 3-5 mins or so. Reauthentication is complete when the new data is compared to previously recently sampled data. Biometrics can come from the collection device as well. The DFA would know the collection device information and can associate the collection CID and the collection device. Mobile biometrics such as face recognition and finger print authentication may also be deployed.

As noted above, components can be used to determine whether CID has been tampered with or removed from an individual, whereby when the product is removed it can be made inoperable. The functionality of the device can also be limited or ceased based on the variables unrelated to the physical state creating a nonphysical frangibility. The device using a combination of electronic components and software can determine when CID has been removed from one person and applied to another. Security components such as holograms may be added in the final step for authentication.

This embodiment allows for the DFA to use the real-time biometrics as triggers for alarms and for making the CID device frangible. To improve battery shelf life and provide an on-off switch can be challenging. This embodiment uses the NFC signal to turn on the microcontroller and can use an RF characteristic to turn off the sensor to preserve battery life. Furthermore, small psychological sensors such as optical, temperature and accelerometers can be added to the microcontroller for the capture of physiological sensors. In a preferred embodiment, for an added layer of security, there would be built in firmware frangibility where all data would be removed from the device, and not allow the device to be used after a set number of hours.

In an alternative embodiment, the authentication may be done using data readings from multiple sensors placed on the body, with the on-body system authentication is performed by a trusted master DFA by the system, reducing the error of having the wrong sensor on a particular person.

The system could be particularly useful in applications relating to taking custody of a person (e.g., a person who has escaped a secure facility or wandered off from a group home). For example, a person may be neurologically impaired and be unwilling or unable to communicate physical distress. In preferred embodiments, DFA would be both identify specific, time specific and location specific while providing continuous vital sign monitoring. In preferred embodiments, the sensor readings would be recorded both locally on the local smart device and in the remote database for both security purposes and quick information delivery. In a preferred embodiment, when the sensor readings indicate concerning vital signs, an alarm/notification would be sent to the officer's smart device, as well as emergency services and the local precinct. Periodically, in response to prompting the system checks data which has been input into the system to determine whether an alarm condition exists. An alarm condition may be also indicated by a complaint which the consumer application downloaded by the user. If an alarm condition is found to exist, the system sends text messages.

The system would be particularly useful during the critical period of time when a person who may be non-communicative or uncooperative. The lack of communication could be due to a number of factors: non-cooperation, inability to speak and/or understand spoken English, mental illness, neurological disorders or developmental or other disabilities. The combination of sensors, a non-transferable device authenticated with an authorized smart device and software with appropriate security protocols provides a system that provides accurate, unalterable data on time, location and vital signs readings. Even without a verified government identification (such as a driver's license), the ability to take a picture of the person would also be time stamped and can be correlated or triangulated with other information.

In accordance with a preferred embodiment of the invention, information stored is retrievable by individuals providing health care at the facility to provide various services, such as, blood pressure measurements, body temperature measurements, administration of nutrition and/or drugs, and so forth. Such prompting may be done by any number of means, such as a handheld mobile device sounding an alarm and presenting an on-screen prompt for a particular service to be provided.

With processing tied to the system rather than a single CID, the use of multiple CIDs could be very useful for first responders in mass injury incident such as a fire, where several people are rescued and get the CID applied and then an alert is sent out to the EMTs in case anyone's vital signs are concerning. This is particularly helpful in situations where there are a number of people injured and the traditional triage process is inefficient. In a preferred embodiment, EMS will be alerted immediately if someone's vital signs show possible distress, and a message could be sent via the central server that additional EMS units are needed.

The DFA and multiple CIDs on person would allow for team sports monitoring in training facilities and on field with a single stream from player to the server for final aggregation and display. This system reduces the number of components that the server has to uniquely pair to an individual and reduce the overall conflicts of multiple persons with multiple sensors. Sensors communication distances could be reduced in signal strength to enable a stronger signal strength while not over polluting the RF environment. Radio transmission is the largest peak current drain on a typical CID. By having a local on body master DFA programed with the sensor specific DFA-CID capabilities, the communication around the body (2 meters) requires less power. To further the communications capability in a sports or warfighter application the secondary or off body communications with the system can be at a higher transmission level for further communications. This also permits the usage of a separate radio protocol, that permits real time location at a higher transmission frequency without causing the CIDs to consume the power.

In a preferred embodiment, a “hierarchical” CID structure could be used to address issues associated with athletes suffering concussions due to field of play head impact. In such an application, two CIDs could be employed in a hierarchical configuration. In this embodiment the one System device (Primary) located on the chest could function as a data collection and retransmission device and contain the Master DFA for all CID communication and authentication and data algorithms, time correlations as described above. The Master DFA may or may not be rechargeable. The secondary CID could be a Mastoid sensor, behind the ear, or in the mouth (Jaw) that is authenticated by the specific DFA to the Master DFA. In operation, sudden head impact information in terms of level of force, direction of impact, and duration of impact could be captured by the Mastoid CID and forwarded to the Master DFA. The Master DFA would then transmit the combined data to the upstream system for subsequent processing, evaluation, and display. In this embodiment a GPS CID configured with accelerometry and appropriate DFA could be developed to acquire real-time motion and position. This sensor would require two DFAs, one for geo-positioning, the second for impact, position, and activity.

In a preferred embodiment, using multiple bio-impedance sensors hydration, lung-function, can be used together. The DFA would provide the intelligence of ground signal drift and enable the Bioimpedance sensors to synchronize the body-system grounding. Each Bio-impedance sensor would have a DFA and a Master DFA would coordinate the two impedance measurements to determine respiration rate, and lung function.

In a preferred embodiment, a combination of a CID capable of measuring temperature and a gateway from a DFA could be combined and applied to monitoring residents in Long Term Care (LTC) facilities. Such residents are particularly susceptible to conditions such as Urinary Tract Infection (UTI). A primary indicator of a UTI infection is elevated temperature. A temperature monitoring CID could be placed on the chest of the resident and continuous temperature monitoring be conducted using a gateway on the DFA, or the DFA could reside as a module in the cloud and the cloud and gateway combination would provide settings, and offload schedules for the sensors. If the resident's temperature exceeded a preset threshold, the medical staff could be notified via the system and remedial medical intervention taken place.

In a preferred embodiment, a “peer-to-peer” CID structure using two CIDs could be used to address issues associated with conditions such as deep vein thrombosis. Individuals suffering from such a condition display a difference in skin temperature between the leg suffering from deep vein thrombosis and a reference temperature such as that present on the chest. Deploying two CIDs on a given subject, collecting the data from each CID via a common DFA function would allow medical staff to continuously monitor for temperature difference thresholds which, when exceeded, would indicate remedial action is needed.

The inventive system can also incorporate or work with external biosensors that can measure skin or core temperature changes, heart rate, hydration, UV Exposure, glucose level (using glucose ink or glucose sensor for example) and use the wireless transmitter to send the information to an external device or reader. The device's application directly to the skin makes it ideal for physiologic applications. These unique sensors can be identified easily to a particular individual by an authorized user scanning an NFC tag and associating the sensor to the individual and activating the DFA to be associated with that individual.

Thus, the system could be used to monitor for basic measurements that are relevant in any setting in or outside of healthcare and can be used as an early indication for medical conditions heat exhaustion, hypothermia, sepsis by measuring biometrics such as salt content, foreign compounds and even dehydration. For monitoring emergency situations (for example in a combat or bioterrorism zone) other sensors such as pressure sensors, radiation sensors, chemical weapons sensors, radiologic sensors, paper sensors may be useful. In some embodiments, the CID could monitor skin temperature changes over time with the probability of developing hypothermia. Conversely, by measuring the changes to core temperature or hydration, the CID can be used as a monitor for heat exhaustion or UV exposure. The data could be saved remotely but not locally.

Sensors may also be incorporated into the device to measure other biometric factors including but not limited to motion, body position, weight, blood pressure, heart rate, cardiac function including but not limited to EKG, blood chemistry such as but not limited to glucose, hydration, or any other physiologic signals, activity, the maximum performance indication, gait analysis or sampled quantitated values. This is particularly advantageous for not only telehealth but also remote monitoring. For example, some COVID-19 patients have silent hypoxia where they are alert and feel relatively well yet have remarkably low blood oxygen saturation levels which can lead to death. By the time they make it to the hospital, the lack of symptoms can make triage extremely difficult. Thus, it would be advantageous for a hospital to be able to scan patient CIDs to properly triage. The CID could be useful as both an identification and monitoring device to measure vital signs such as pulse oximetry and heart rate to alert authorities to health distress in the field. Furthermore, being able to compute maximal performance in individuals and track that from everyday life activities can show both positive and negative impacts of daily living on the physiology of the patient.

In an alternative embodiment the CID can be placed in a visible area such as the hand, forearm, or shoulder and paired to biometric measuring device(s) placed in an area more conducive to accurate measurements. This allows biometric CIDs to be placed in the most effective places for biometric measurements while an Identification the DFA-CID is more easily accessible for scanning or more appropriately placed for transmitting a signal. When using biometric sensing, the lack-of-physiological sensor data is an indication of the sensor not being worn, or being transferred and both the sensor and the cloud based system can deactivate the sensor, provide alarms to the caregiver and system.

In another embodiment, the system may comprise multiple CIDs can communicate with each other via the DFA wirelessly and transmit information wirelessly and process third party information as well. This data can be transferred when any one of the CIDs are scanned or the data can be transmitted wirelessly to any device which can read the receive the signal wirelessly. Receiving devices can include gateways, smart devices and smart home devices.

The combination of the non-transferable CID married to a central system, means that any data, information and measurements taken are updated in local and central databases. The central server can send notices (alarms, notices, follow up reminders and check-ins and the like), thus putting in a process in place to keep the wearer (and/or their designated caregiver) well apprised of any vital information that may need to be communicated.

The advantages of an integrated system with a cutaneous identification device which accurately identifies and verifies their location becomes acutely evident during situations where tracking and tracing are a must. During an outbreak, epidemic or pandemic, in addition to secure identification, tracking and contract tracing becomes a high priority to mitigate the spread of disease. This system enables accurate location tracking, contact tracing and, in preferred embodiments with Bluetooth sensors all the time. By having a system track CID wearers' locations and potential interactions with others, given their geolocations, make notifications more efficient and accurate. This is vastly improved from a smartphone system without a CID. This pairing of the CID with the smartphone means that the locations can be accurately traced. By having the CID scanned at various locations (stores, doctor's office, work, gym, etc.), there is a record of time and location and also proximity to others.

Additionally, with an option to measure biometrics through other paired devices, it is possible to have a wearer's symptoms tracked over time. There could also be a requirement to self-report symptoms. As discussed above, in addition to temperature, other sensors can be added that can detect or measure for heat exhaustion, hypothermia, sepsis by measuring biometrics such as salt content, foreign compounds and even dehydration UV exposure, skin temperatures changes over time, glucose, salt content, blood alcohol level or detect the presence of other substances. Such measurements and tracking can be particularly to medical professionals in determining diagnosis and treatment plans in an urgent situation.

In an alternative embodiment, when vital sign data is concerning, the system through preprogrammed settings can identify the individual and connect to emergency response systems and provide a mechanism by which identification is verified and critical information is made available to emergency responders for the right person at the right time. Current systems cannot distinguish between the person calling emergency services and the person in need of assistance because they rely on the phone as the main identification point. The system's CID paired with the DFA's can serve as the verification point to allow emergency response access to databases which contain detailed information that could be useful in an emergency such as allergies, emergency contact, proxy, medical insurance info, et

This would not only be useful in general emergency situations, but also particularly useful in situations such as camping. These workflows include sending first responders a map of camper's geolocation, sending onsite personnel camper's medical information (allergies, medicines, insurance card and release form) and sending camper's parents/guardians a text with the phone number of onsite medical personnel. If emergency services button is not clicked, camp staff can view the camper's emergency contacts, medical records and release form as well as submit an incident report.

Additionally, users can their CID in conjunction to access their user portal and view certain information drawn from their admission and medical records. After discharge, users can scan their CID to access their discharge information and view their ER diagnosis, expected course of illness, self-care instructions and return precautions.

The system can also send alerts to someone to be aware of their systems and go to the hospital if they have certain other symptoms, making the system function as individual specific care as opposed to a generic blast. Outside of a pandemic, the tracking and tracing functions are particularly helpful to support public health in general to be aware of smaller outbreaks and to send out alerts, so people are acutely aware of their symptoms. In a preferred embodiment, there would be self-reporting of behavior (handwashing, gargling) and tracking of the same to see how timely information can shape individual behavior and public health outcomes.

While illustrative embodiments of the invention have been described, it is noted that various modifications will be apparent to those of ordinary skill in the art in view of the above description and drawings. These are within the scope of the present invention which is limited and defined only by the following claims.

1. Apparatus for measuring temperature on a mammal such as a human, comprising:
(a) a temperature measurement device, comprising:
(i) an onboard device power source powering components of said temperature measurement device;
(ii) a clock having a first high speed output, and a second low speed output, said high speed output being at least 10 times as fast as said low speed output;
(iii) a data memory sector;
(iv) a digital processor responsive to said high-speed output to generate a storage trigger signal;
(v) a temperature measurement transducer responsive, to said storage trigger signal from said digital processor, to periodically collect a temperature measurement and to couple said collected temperature measurement to said data memory sector to store said collected temperature measurement in said data memory sector to accumulate data in said data memory sector as an accumulation of data in the form of a plurality of data points;
(vi) a wireless transceiver having an input and an output, said digital processor responsive to said low speed output to generate a transmission trigger signal and couple said transmission trigger signal to said data memory sector, said transmission trigger signal causing said data memory sector, to couple said accumulation of data stored in said data memory sector to the input of said wireless transceiver for transmission; and
(vii) a non-volatile memory sector with a program of onboard instructions for controlling said digital processor to cause said temperature measurement transducer to store said data points in said data memory sector to cause said wireless transceiver to transmit said data points; and
(viii) a chassis member supporting said power source, said clock, said data memory sector, said digital processor, said temperature measurement transducer and said wireless transceiver;
(b) a publicly accessible network;
(c) a wireless repeater receiving the accumulation of data output from said wireless transceiver and coupling the same to said publicly accessible network;
(d) a server coupled to said publicly accessible network to receive said accumulation of data, said server comprising:
(i) a server central processing unit;
(ii) a temperature data memory coupled to receive and store said data points; and
(iii) a non-volatile server program memory with a program of server instructions causing said server central processing unit to receive, store, and transmit to at least one user said data points; and
(e) a non-volatile temperature conversion program memory with a program of temperature conversion instructions for converting said data points to predicted physiological temperature readings for said transmission to said at least one user wherein said non-volatile temperature conversion program memory is not located on said chassis or powered by said onboard device power source.

2. Apparatus as in claim 1, wherein said predicted physiological temperature is a predicted oral temperature, and further comprising an adhesive member for securing said chassis member to the chest of a patient.

3. Apparatus as in claim 1, wherein said predicted physiological temperature is a predicted oral temperature, and wherein said transceiver is a mobile personal computing device coupled to said publically accessible network, and the non-volatile temperature conversion program memory is coupled to and runs on said personal computing device.

4. Apparatus as in claim 1, wherein said wireless repeater is a smartphone and the non-volatile temperature conversion program memory is coupled to and runs on a digital signal processor on said smartphone.

5. Apparatus as in claim 1, wherein said predicted physiological temperature is a predicted oral temperature, and wherein said chassis member is a waterproof housing.

6. Apparatus as in claim 1, wherein the non-volatile temperature conversion program memory is coupled to and runs on said server central processing unit.

7. Apparatus for measuring temperature, comprising:
(a) a temperature measurement device, comprising:
(i) a device power source powering components of said temperature measurement device; dependent battery or capacitor
(ii) a clock having a first high speed output, and a second low speed output, said high speed output of said clock being at least 10 times as fast as said low speed output;
(iii) a data memory sector;
(iv) a digital processor responsive to said high-speed output to generate a storage trigger signal;
(v) a temperature measurement transducer responsive, to said storage trigger signal from said digital processor, to periodically collect a temperature measurement and to couple said collected temperature measurement to said data memory sector to store said collected temperature measurement in said data memory sector to accumulate data in said data memory sector as an accumulation of data in the form of a plurality of data points;
(vi) a wireless transceiver having an input and an output, said digital processor responsive to said low speed output to generate a transmission trigger signal and couple said transmission trigger signal to said data memory sector, said transmission trigger signal causing said data memory sector, to couple said accumulation of data stored in said data memory sector to the input of said wireless transceiver for transmission; and
(vii) a non-volatile memory sector with a program of onboard instructions for controlling said digital processor to cause said temperature measurement transducer to store said data points in said data memory sector to cause said wireless transceiver to transmit said data points; and
(viii) a chassis member supporting said power source, said clock, said data memory sector, said digital processor, said temperature measurement transducer and said wireless transceiver;
(b) a publicly accessible network;
(c) a wireless repeater receiving the accumulation of data output from said wireless transceiver and coupling the same to said publicly accessible network;
(d) a server coupled to said publicly accessible network to receive said accumulation of data, said server comprising:
(i) a server central processing unit;
(ii) a temperature data memory coupled to receive and store said data points; and
(iii) a non-volatile server program memory with a program of server instructions causing said server central processing unit to receive, store, and transmit to at least one user said data points; and
(e) a non-volatile temperature conversion program memory with a program of temperature conversion instructions for converting said data points to predicted physiological temperature readings for said transmission to said at least one user, wherein said program of temperature conversion instructions for converting said data points to predicted physiological temperature readings was generated by collecting a plurality of collected clinical calibration skin temperature measurements from skin mounted temperature measurement transducers, and collecting a plurality of collected clinical calibration physiological temperature measurements, said calibration skin temperature measurements and said clinical calibration physiological temperature measurements comprising a plurality of data points, and fitting a function to said plurality of data points.
